(19) United States
(12) Patent Application Publication
(10) Pub. No.: US 2021/0282795 A1
(43) Pub. Date: Sep. 16, 2021

(54) MEDICAL GRIPPING DEVICE

(71) Applicants: KANAGAWA INSTITUTE OF INDUSTRIAL SCIENCE AND TECHNOLOGY, Ebina-shi, Kanagawa (JP); KEIO UNIVERSITY, Minato-ku, Tokyo (JP)

(72) Inventors: Tomoyuki SHIMONO, Kanagawa (JP); Hikaru SASAKI, Tokyo (JP); Kouhei OHNISHI, Kanagawa (JP); Shunsuke SHIBAO, Tokyo (JP); Takahiro MIZOGUCHI, Kanagawa (JP); Takuya MATSUNAGA, Kanagawa (JP); Eriko ABIKO, Tokyo (JP); Masaaki NISHIMOTO, Tokyo (JP); Mika AOKI, Kanagawa (JP)

(73) Assignees: KANAGAWA INSTITUTE OF INDUSTRIAL SCIENCE AND TECHNOLOGY, Ebina-shi, Kanagawa (JP); KEIO UNIVERSITY, Minato-ku, Tokyo (JP)

(21) Appl. No.: 17/258,075

(22) PCT Filed: Jun. 7, 2019
(86) PCT No.: PCT/JP2019/022778

§ 371 Date: Jan. 5, 2021

(30) Foreign Application Priority Data
Jul. 6, 2018 (JP) 2018-129425

(51) Int. Cl. A61B 17/29; A61B 34/37

(52) U.S. Cl. CPC A61B 17/29; A61B 2017/00367; A61B 2017/2902; A61B 34/37

(57) ABSTRACT

A medical gripping device includes a housing, a gripping mechanism at one end of the housing, an operation unit, and a reaction force actuator and a gripping actuator installed in the housing. The operation unit is operated by a gripping operation of an operator, and the reaction force actuator applies an operation reaction force to the operation unit. The gripping actuator causes the gripping mechanism to perform a gripping operation. A control unit controls a force and a position that are output by the gripping actuator in an operation of the gripping mechanism in accordance with an operation with respect to the operation unit, and controls a force and a position that are output by the reaction force actuator in an operation of applying the operation reaction force to the operation unit in accordance with a reaction from a gripped object with respect to the gripping mechanism.

### TECHNICAL FIELD

The present invention relates to a medical gripping device that has the function of forceps.

### BACKGROUND ART

A medical master-slave remote surgical device exhibits high performance in minimally invasive surgery, and devices that are typified by da Vinci surgical systems (trademark) have already been put into practical use.

As disclosed in Patent Literature 1 and Patent Literature 2, medical forceps devices having a master-slave structure are also known.

### CITATION LIST

### Patent Literature

[PTL 1] International Publication No. 2005/109139

[PTL 2] International Publication No. 2015/041046

### SUMMARY OF INVENTION

### Technical Problem

However, a conventional medical master-slave remote surgical device has problems in that, for example, the device is large and training is required to use the device. The device does not provide tactile-force feedback, and thus is difficult to apply in the field of, for example, neurosurgical procedure that requires delicate work. On the other hand, since the medical forceps devices described in Patent Literature 1 and Patent Literature 2 have the form of forceps, it is highly probable that their purpose of use is limited to the conventional range of use of forceps.

In contrast, in, for example, neurosurgical procedure, smaller forceps (tweezers) that allow an operator to more directly feel a haptic sensation is used. Therefore, medical devices capable of being used similarly to conventional forceps and having more functions than such conventional forceps have not been realized.

It is an object of the present invention to realize a medical gripping device that can be used as forceps and has more functions than forceps.

### Solution to Problem

In order to achieve the object described above, a medical gripping device according to an aspect of the present invention comprising:

an operation unit that is operated by a gripping operation of an operator;

a first actuator that applies an operation reaction force to the operation unit;

a gripping unit that grips a grip object;

a second actuator that causes the gripping unit to perform a gripping operation;

a housing that has the gripping unit at one end and the operation unit between the one end and the other end, the first actuator and the second actuator being installed in the housing; and

a control unit that controls a force and a position that are output by the second actuator in an operation of the gripping unit in accordance with an operation with respect to the operation unit, and controls a force and a position that are output by the first actuator in an operation of applying the operation reaction force to the operation unit in accordance with a reaction from the grip object with respect to the gripping unit.

### Advantageous Effects of Invention

According to the present invention, it is possible to realize a medical gripping device that can be used as forceps and has more functions than forceps.

### BRIEF DESCRIPTION OF DRAWINGS

FIG. 1 is a schematic view of a basic structure of a medical gripping device 1 according to the present invention.

FIG. 2A is a schematic view of a mechanism on a master side of the medical gripping device 1.

FIG. 2B is a schematic view of a mechanism (another example) on the master side of the medical gripping device 1.

FIG. 3 is a schematic view of a mechanism on a slave side of the medical gripping device 1.

FIG. 4 is a block diagram of bilateral control used in the present invention.

FIG. 5 is a schematic view of a first example of a device structure of the medical gripping device 1 according to the present invention.

FIG. 6 is a schematic side view of the medical gripping device 1 in the first example of the device structure.

FIG. 7 is a schematic top view of the medical gripping device 1 in the first example of the device structure.

FIG. 8 is a schematic view of a modification of the first example of the device structure.

FIG. 9 is a schematic side view of the medical gripping device 1 in the modification of the first example of the device structure.

FIG. 10 is a schematic top view of the medical gripping device 1 in the modification of the first example of the device structure.

FIG. 11 is a schematic view of a second example of a device structure of the medical gripping device 1 according to the present invention.

FIG. 12 is a schematic side view of the medical gripping device 1 in the second example of the device structure.

FIG. 13 is a schematic top view of the medical gripping device 1 in the second example of the device structure.

FIG. 14 is a schematic view of a modification of the second example of the device structure.

FIG. 15 is a schematic side view of the medical gripping device 1 in the modification of the second example of the device structure.

FIG. 16 is a schematic top view of the medical gripping device 1 in the modification of the second example of the device structure.

FIG. 17 is a schematic view of experimental conditions of Experiment 1.

FIG. 18A shows time response measurement results of the positions of an actuator on the master side and an actuator on the slave side.

FIG. 18B shows inverse values of a reaction force that a slave gripping actuator 50 is subjected to from an environment.

FIG. 19 shows changes in reaction force with respect to displacement of a gripping mechanism 40 in Experiment 1.

FIG. 20 is a schematic view of experimental conditions of Experiment 2.

FIG. 21A shows the results of an experiment in which samples are gripped without performing scaling.

FIG. 21B shows the results of an experiment in which samples are gripped by performing scaling in which a force is doubled.

### DESCRIPTION OF EMBODIMENTS

Embodiments of the present invention are described below by using the drawings.

### [Basic Concept of the Present Invention]

A medical gripping device according to the present invention has a structure in which a reaction force actuator and a gripping actuator are integrated with each other in a housing. The reaction force actuator applies an operation reaction force to an operation unit that is operated by a gripping operation of an operator. The gripping actuator causes a gripping mechanism that grips a grip object to perform a gripping operation. In an example, in the medical gripping device, the operation unit is provided in a central portion of the housing, and the gripping mechanism is provided at a distal end portion of the housing. When the operator operates the operation unit by the gripping operation (operation for holding the grip object by forceps), based on information about a force and a position that have been input to the operation unit, the gripping actuator outputs a force (a gripping force) and a position (a gripping amount) for causing the gripping mechanism to perform the gripping operation in accordance with the operation that has been input to operation unit. In addition, at this time, based on information about a force and a position resulting from a reaction that is received from the grip object, the reaction force actuator outputs a force and a position for performing an operation of applying the operation reaction force at the operation unit. That is, bilateral control in which the reaction force actuator is a master actuator and the gripping actuator is a slave actuator is performed.

Therefore, the operator can operate, with a feeling of use that is similar to that of forceps of medical instruments, the medical gripping device of a form that is similar to forceps constituted by a master-slave device.

Since the gripping operation is realized by the bilateral control, the medical gripping device can be provided with functions that are not provided by forceps of medical instruments, such as performing scaling of a force or a position or indicating the hardness of a grip object in numerical form.

That is, according to the present invention, it is possible to realize a medical gripping device that can be used as forceps and has more functions than forceps.

A structure of the medical gripping device according to the present invention is described below.

### [Basic Structure]

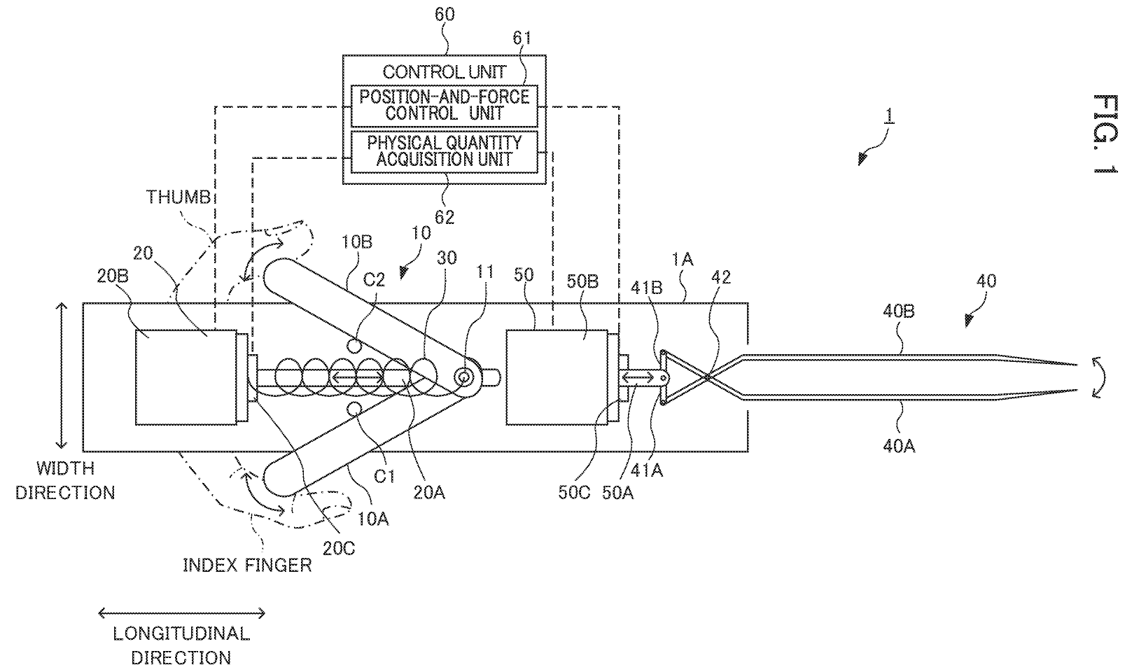

FIG. 1 is a schematic view of a basic structure of a medical gripping device 1 according to the present invention.

Note that FIG. 1 is a schematic top view of the medical gripping device 1, and shows an internal structure of the medical gripping device 1 that can be seen through a housing 1A. In addition, in FIG. 1, a state of a hand of an operator that operates the medical gripping device 1 is schematically shown by alternate long and short dashed lines.

As shown in FIG. 1, the medical gripping device 1 includes an operation unit 10, a reaction force actuator 20, a restoring spring 30, a gripping mechanism 40, a gripping actuator 50, and a control unit 60. The components other than the control unit 60 are installed in the housing 1A. However, the control unit 60 may be installed in the housing 1A. The arrangement of each component shown in FIG. 1 is an example, and other forms of arrangements of each component are possible as long as the function of the medical gripping device 1 can be realized. Further, the gripping mechanism 40, being a straight-type gripping mechanism as shown in FIG. 1, may also be a bayonet-type gripping mechanism. Note that electric power is supplied to the medical gripping device 1 from a battery (not shown) included therein or from an external power source.

In the structure shown in FIG. 1, a portion including the operation unit 10, the reaction force actuator 20, and the restoring spring 30 is a master side, and a portion including the gripping mechanism 40 and the gripping actuator 50 is a slave side.

The operation unit 10 includes a pair of levers 10A and 10B that are rotatably connected at one end, and a rotation shaft 11 that is connected to the levers 10A and 10B is installed so as to be movable in a longitudinal direction of the housing 1A. At the operation unit 10, the other end of the lever 10A protrudes from a through hole formed in one side portion of the housing 1A, and the other end of the lever 10B protrudes from a through hole formed in the other side portion of the housing 1A. Inner side walls of the corresponding levers 10A and 10B (side walls facing the reaction force actuator 20) are in contact with corresponding circularly cylindrical members C1 and C2 fixed to the housing 1A. At the operation unit 10, when a gripping operation has been performed and when being restored from the gripping operation, while the rotation shaft 11 moves in the longitudinal direction of the housing 1A and the inner side walls of the levers 10A and 10B are in sliding contact with the corresponding circularly cylindrical members C1 and C2, an opening-closing state of the levers 10A and 10B changes.

The reaction force actuator 20 is constituted by a small, high-output motor, such as a voice coil motor. A mover 20A is installed so as to be movable in the longitudinal direction of the housing 1A, and a stator 20B is fixed to the housing 1A. The position of the mover 20A is detected by a position sensor 20C, such as a linear encoder. A distal end of the mover 20A of the reaction force actuator 20 is connected to the rotation shaft 11 of the operation unit 10. Therefore, the reaction force actuator 20 is capable of controlling the movement of the rotation shaft 11 in the longitudinal direction of the housing 1A. That is, the reaction force actuator 20 is capable of performing an operation of applying an operation reaction force with respect to the operation of the levers 10A and 10B.

One end of the restoring spring 30 is connected to the stator 20B of the reaction force actuator 20 (or the housing 1A) and the other end of the restoring spring 30 is connected to the rotation shaft 11 at the levers 10A and 10B. The restoring spring 30 has a natural length when the connection angle between the levers 10A and 10B is the largest (maximally open state), and is brought into a stretched state the more the levers 10A and 10B are operated. Therefore, when the operation of the levers 10A and 10B is stopped, an elastic force of the restoring spring 30 causes the restoring spring 30 to return to the position of natural length and thus causes the levers 10A and 10B to be restored to the maximally open state. However, it is possible to provide a structure in which a spring corresponding to the restoring spring 30 is installed on the slave side and, when the operation is stopped, the gripping mechanism 40 is restored to the maximally open state.

Note that it is possible to realize the elastic force of the restoring spring 30 by an output of the reaction force actuator 20, in which case, a structure that does not include the restoring spring 30 can be used. In addition, elastic bodies other than springs may be used to realize effects that are the same as those of the restoring spring 30.

The gripping mechanism 40 includes a pair of gripping members 40A and 40B that correspond to distal end portions of forceps of medical instruments. The distal ends of the corresponding gripping members 40A and 40B protrude from one end of the housing 1A and grip tissues of a grip object at the time of a surgical operation. The other end sides of the corresponding gripping members 40A and 40B are bent in directions in which they approach and intersect each other, and, at an intersection portion, are rotatably connected to each other by a rotation shaft 42. Note that the rotation shaft 42 is fixed to the housing 1A or a portion that is integrated with the housing 1A (for example, a reinforcing member). Further, the other ends of the corresponding gripping members 40A and 40B are rotatably connected to one ends of corresponding link members 41A and 41B. The other ends of the corresponding link members 41A and 41B are rotatably connected at a distal end of a mover 50A of the gripping actuator 50.

The gripping actuator 50 is constituted by a small, high-output motor, such as a voice coil motor. The mover 50A is installed so as to be movable in the longitudinal direction of the housing 1A, and a stator 50B is fixed to the housing 1A. The position of the mover 50A is detected by a position sensor 50C, such as a linear encoder. The distal end of the mover 50A of the gripping actuator 50 is connected to a connection portion at which the link members 41A and 41B are connected to each other (a rotation shaft). Due to the gripping actuator 50 moving the mover 50A, the connection angle between the link members 41A and 41B is changed, and thus the relationship between the positions of the other ends of the corresponding gripping members 40A and 40B changes. Since the gripping members 40A and 40B are rotatably connected to each other at the rotation shaft 42 fixed to the housing 1A, when the relationship between the positions of the other ends of the corresponding gripping members 40A and 40B changes, the distal ends of the corresponding gripping members 40A and 40B undergo an opening-closing operation. Specifically, when the other ends of the gripping members 40A and 40B approach each other, they operate in a direction in which the distal ends of the corresponding gripping members 40A and 40B close, and, when the other ends of the corresponding gripping members 40A and 40B move away from each other, they operate in a direction in which the distal ends of the corresponding gripping members 40A and 40B open. That is, due to the gripping actuator 50 moving the mover 50A, in the gripping mechanism 40, the gripping members 40A and 40B are capable of undergoing the opening-closing operation.

The control unit 60 is constituted by an information processing device such as a microcomputer or a LSI (Large-Scale Integrated Circuit), and, based on the positions detected by the position sensors 20C and 50C, transmits, by performing bilateral control, a haptic sensation between the reaction force actuator 20 and the gripping actuator 50. Based on the information acquired by the bilateral control, the control unit 60 acquires a physical quantity (such as hardness) of a grip object.

Specifically, the control unit 60 includes a position-and-force control unit 61 that controls a position and a force and a physical quantity acquisition unit 62 that acquires a physical quantity of a grip object.

When an operator has operated the operation unit 10 by a gripping operation, the position-and-force control unit 61 acquires a detection value of a position to which the mover 20A of the reaction force actuator 20 has moved. The position-and-force control unit 61 calculates the positions of the levers 10A and 10B (operation amounts) from the detected positions, calculates the acceleration of the mover 20A, and, from the calculated acceleration, calculates a force (an operation force) that has been input to the operation unit 10. Then, the position-and-force control unit 61 controls the output of the gripping actuator 50 so as to reproduce the gripping amount and the gripping force corresponding to the positions (operation amounts) and the forces (operation forces) of the levers 10A and 10B. At this time, the position-and-force control unit 61, by using a parameter that is in accordance with the mechanical structure of the gripping mechanism 40, calculates target values of the positions (the gripping amounts) and the forces (gripping forces) of the gripping members 40A and 40B, and outputs command values (such as electrical-current command values) that are in accordance with the calculated target values to the gripping actuator 50. Therefore, a gripping operation corresponding to the operation of the operation unit 10 is realized at the gripping mechanism 40.

When the gripping mechanism 40 is to perform the gripping operation, the position-and-force control unit 61 acquires a detection value of a position to which the mover 50A of the gripping actuator 50 has moved. The position-and-force control unit 61 calculates the positions of the gripping members 40A and 40B (gripping amounts) from the detected position, calculates the acceleration of the mover 50A, and, from the calculated acceleration, calculates a force (reaction force) that has been input to the gripping members 40A and 40B. Then, the position-and-force control unit 61 controls the output of the reaction force actuator 20 so as to reproduce a state of reaction corresponding to the positions (gripping amounts) and the forces (reaction forces) of the gripping members 40A and 40B. At this time, the position-and-force control unit 61, by using a parameter that is in accordance with the mechanical structure of the operation unit 10, calculates target values of the positions (the operation amounts) and the forces (reaction forces) of the levers 10A and 10B, and outputs command values (such as electrical-current command values) that are in accordance with the calculated target values to the reaction force actuator 20. Therefore, the operation amounts and the reaction forces that are in accordance with the state of reaction of the gripping mechanism 40 are realized at the operation unit 10.

The physical quantity acquisition unit 62 acquires data about the hardness of the grip object from the parameters that are acquired by the bilateral control. Specifically, from an estimated value of the reaction force that is generated when the grip object is gripped by the gripping mechanism 40, the physical quantity acquisition unit 62 calculates the data about the hardness of the grip object. Note that the physical quantity acquisition unit 62 can be constituted by, for example, a reaction force estimation observer.

Note that, when the restoring spring 30 is installed in the medical gripping device 1, the elastic force of the restoring spring 30 (the force for restoring to the maximally open state) acts upon the operation unit 10. Therefore, when the control unit 60 performs the bilateral control, the control unit 60 is capable of performing the control so that the elastic force of the restoring spring 30 that changes in accordance with the positions of the levers 10A and 10B is calculated, and the calculated elastic force is subtracted to add the operation reaction force.

By causing the control unit 60 to control the reaction force actuator 20 with respect to the spring constant that the restoring spring 30 physically has, it is possible to feel a spring having a large spring constant or a spring having a small spring constant.

Therefore, it is possible to adjust the medical gripping device 1 to a state that is easily operable by an operator, and to realize high operability thereof.

When the medical gripping device 1 having such a structure is to be operated, an operator operates the medical gripping device 1 with the index finger and the thumb gripping the levers 10A and 10B that are held between the index finger and the thumb and with the medical gripping device 1 being placed on the back of the hand (first interdigital space).

Therefore, with respect to the position at which the operation unit 10 is installed, it is suitable to set the center of gravity of the medical gripping device 1 on the other end side opposite to one end at which the gripping mechanism 40 is provided.

Due to such a structure, the operator is less likely to feel the weight of the medical gripping device 1 and the operability of the medical gripping device 1 can be increased.

The housing 1A may have a structure having a shape that easily fits the shape of a hand of an operator by forming a recess in a bottom surface side of the operation unit 10, the recess receiving the back of the hand (the first interdigital space) of the operator.

### [Kinematics of Medical Gripping Device 1]

Next, the kinematics of the medical gripping device 1 is described.

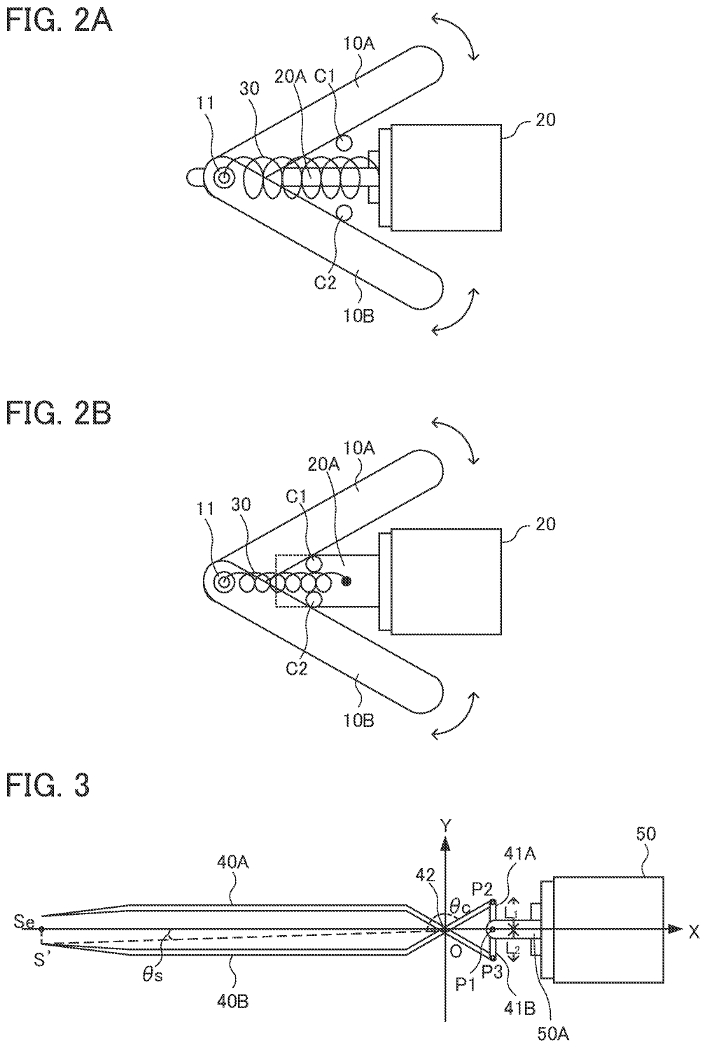

FIG. 2A is a schematic view of a mechanism on the master side of the medical gripping device 1.

As shown in FIG. 2A, on the master side of the medical gripping device 1, the restoring spring 30 is installed at a connection portion at which the reaction force actuator 20 and the levers 10A and 10B are connected to each other, and the restoring spring 30 constantly applies a force in a direction in which the levers 10A and 10B open.

Note that other structures can be used as long as, as with the mechanism shown in FIG. 2A, such structures are capable of applying an operation reaction force and the levers 10A and 10B can be restored to their maximally open state at the time of non-operation.

FIG. 2B is a schematic view of a mechanism (another example) on the master side of the medical gripping device 1.

In the example shown in FIG. 2B, the rotation shaft 11 shown in FIG. 1 is fixed to the housing 1A, and the circularly cylindrical members C1 and C2 are installed at the mover 20A of the reaction force actuator 20. One end of the restoring spring 30 is connected to the mover 20A of the reaction force actuator 20, and the other end of the restoring spring 30 is connected to the rotation shaft 11 at the levers 10A and 10B. The inner side walls of the corresponding levers 10A and 10B (the side walls facing the reaction force actuator 20) are in contact with the corresponding circularly cylindrical members C1 and C2 at which the mover 20A of the reaction force actuator 20 is installed. At the operation unit 10, when a gripping operation has been performed and when being restored from the gripping operation, by moving the mover 20A in the longitudinal direction of the housing 1A while the inner side walls of the levers 10A and 10B are in sliding contact with the corresponding circularly cylindrical members C1 and C2, an opening-closing state of the levers 10A and 10B changes.

Even in the example shown in FIG. 2B, the restoring spring 30 has a natural length when the connection angle between the levers 10A and 10B is the largest (maximally open state), and is brought into a stretched state the more the levers 10A and 10B are operated. Therefore, when the operation of the levers 10A and 10B is stopped, an elastic force of the restoring spring 30 causes the restoring spring 30 to return to the position of natural length and thus causes the levers 10A and 10B to be restored to the maximally open state.

FIG. 3 is a schematic view of a mechanism on the slave side of the medical gripping device 1.

As shown in FIG. 3, on the slave side of the medical gripping device 1, the gripping members 40A and 40B are connected to the gripping actuator 50 via a pair of slider-crank mechanisms, and linear motion of the gripping actuator (the mover 50A) is converted into rotational motion of the gripping members 40A and 40B.

As shown in FIG. 3, an X axis and a Y axis are set and the position of the rotation shaft 42 is an origin O. In FIG. 3, the length of the link member 41A is L₁, the length of the link member 41B is L₂, a connection point at which the link members 41A and 41B and the mover 50A are connected to each other is a point P1, a connection point at which the gripping member 40B and the link member 41A are connected to each other is a point P2, a distal end of the gripping member 40B is a point S′, a point of intersection of a line that is extended perpendicularly from the point S′ with the X axis is a point θe, and the angle between a line segment OS′ and a line segment OP2 is θc.

As a result, the relationship between a position xₛ at the point P1, a rotation angle θs of the gripping member 40B (an angle between the X axis and the line segment OS′), and the angle θc between the line segment OS′ and the line segment OP2 is expressed by the following formula.

$$\begin{matrix}[ Math . 1 ] & \\ {θ}_{S} = arccos ( - \frac{{L}_{1}^{2} - {L}_{2}^{2} + {x}_{S}^{2}}{2 {L}_{2} {x}_{S}} ) - ( π - {θ}_{C} ) & ( 1 )\end{matrix}$$

The relationship between a speed xₛ′ of the gripping actuator 50 and an angular speed θs′ of the gripping member 40B can be obtained by differentiating both sides of Formula (1) by time t.

$$\begin{matrix}[ Math . 2 ] & \\ {\dot{θ}}_{S} = {J}_{x} {\dot{x}}_{S} & ( 2 ) \\ {J}_{x} = - \frac{{x}_{S}^{2} + {L}_{1}^{2} - {L}_{2}^{2}}{{x}_{S} \sqrt{4 {L}_{2}^{2} {x}_{S}^{2} - {( {L}_{1}^{2} - {L}_{2}^{2} - {x}_{S}^{2} )}^{2}}} & ( 3 )\end{matrix}$$

Note that since the maximum value of θs is sufficiently small, it is possible to use an approximation formula of Formula (4).

[Math. 3]

SₑS′=OS′θₛ≈OS′ sin θₛ=yₑₙᵥ (4)

Due to Formula (4), a displacement yₑₙᵥ of the distal end of the gripping member 40B can be considered as only a component in a Y-axis direction.

Since the pair of slider-crank mechanisms are formed by superimposing upon each other two mechanisms that are the same, the relationship between an output torque τ0 at the origin O and a reaction force Fₑₙᵥ to which distal end portions of the corresponding gripping members 40A and 40B are subjected from an environment can be expressed by Formulas (5) and (6).

τ0=(½Jx)Fs (5)

Fₑₙᵥ=τ0/OS′ (6)

Note that, as on the master side, other structures can be used for the slave-side mechanism as long as a gripping operation that is the same as the gripping operation of the mechanism shown in FIG. 3 can be performed.

### [Bilateral Control]

Next, bilateral control that is used in the present invention is described.

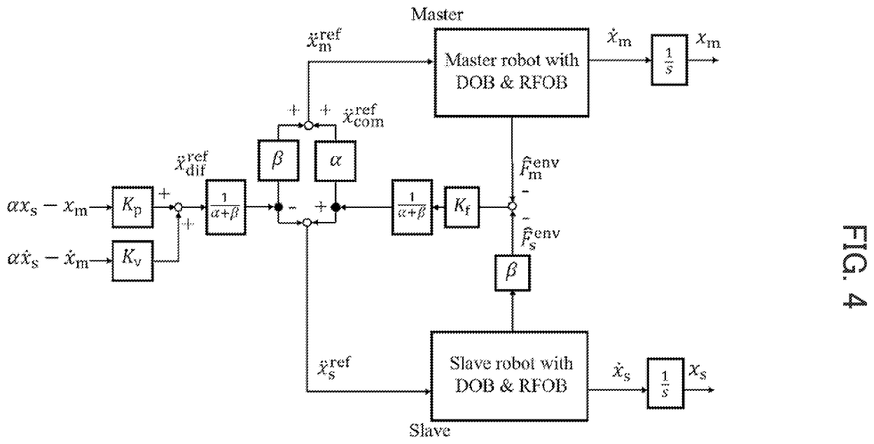

FIG. 4 is a block diagram of the bilateral control used in the present invention.

Note that, in FIG. 4, Kp is a gain in position, Kv is a gain in speed, Kf is a gain in force, a subscript env stands for an input from an environment, a subscript m stands for a master parameter, a subscript s stands for a slave parameter, a subscript ref stands for a reference value (standard value), a subscript com stands for cumulative, a subscript dif stands for differential, and \hat{ } stands for an estimated value.

In FIG. 4, DOB (Disturbance OBserver) compensates for disturbance that is input to an actuator of a master robot and an actuator of a slave robot, and a RFOB (Reaction Force OBserver) estimates the reaction force Fₑₙᵥ received from an environment.

Control target values of positions and the forces in a bilateral control method shown in FIG. 4 satisfy Formulas (7) and (8).

xₘ−xₛ=0 (7)

Fₘ+Fₛ=0 (8)

Note that, in Formulas (7) and (8), xₘ is the position of the master robot, xₛ is the position of the slave robot, Fₘ is a force that is output by the master robot, and Fₛ is a force that is output by the slave robot.

Formula (7) means that the position of the actuator of the master robot and the position of the actuator of the slave robot follow each other, and Formula (8) means that the force that is output from the master robot and the force that is output from the slave robot satisfy the action-reaction law. By satisfying these formulas at the same time, the bilateral control of the present invention realizes the transmission of haptic sensation.

Further, in the bilateral control method of the present invention, it is possible to increase/decrease movements (scaling of positions or forces) while causing the positions and the forces to follow each other.

Here, the control target values are expressed by Formulas (9) and (10).

Xₘ=αXₛ (9)

Fₘ=−βFₛ (10)

Here, any real positive numbers can be used as α and β.

Therefore, it is possible to increase/decrease the haptic sensation and to transmit a larger haptic sensation or a smaller haptic sensation.

### [Specific Examples of Device Structures]

Next, examples of specific device structures of the medical gripping device 1 according to the present invention are described.

### [First Example of Device Structure]

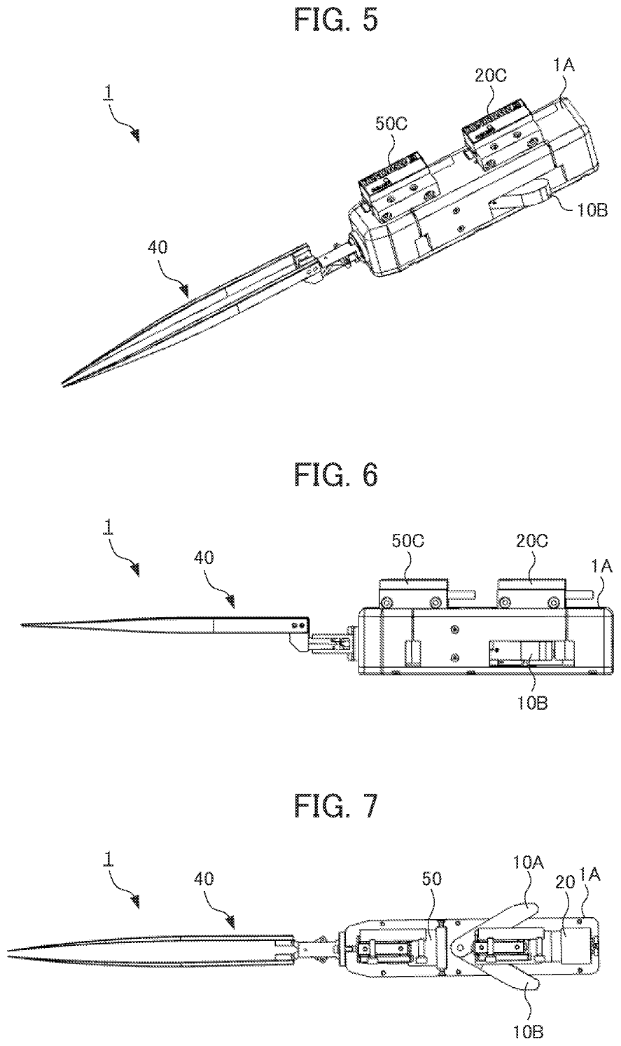

FIG. 5 is a schematic view of a first example of a device structure of the medical gripping device 1 according to the present invention.

Note that FIG. 5 shows an external structure (is a perspective view) of the medical gripping device 1 according to the first example of the device structure.

FIG. 6 is a schematic side view of the medical gripping device 1 in the first example of the device structure. FIG. 7 is a schematic top view of the medical gripping device 1 in the first example of the device structure.

Note that FIG. 7 shows a main internal structure that can be seen through an upper surface of the housing 1A.

As shown in FIGS. 5 to 7, in the first example of the device structure, the gripping mechanism 40 is installed at one end of the housing 1A, and the gripping actuator 50 is adjacently installed at a side of the one end in the housing 1A. In the housing 1A, with respect to the gripping actuator 50, the operation unit 10 is disposed on the other end side opposite to the one end at which the gripping mechanism 40 is provided, and the reaction force actuator 20 is installed further toward the other end side. Note that the first example of the device structure shown in FIGS. 5 to 7 is an example in which the gripping mechanism 40 having a distal-end shape that is the same as that of straight-type forceps of is provided.

In the case of such a structure, since the structure on the master side and the structure on the slave side can be reduced in size, it is possible to reduce the weight and the size of the medical gripping device 1.

### [Modification of First Example of Device Structure]

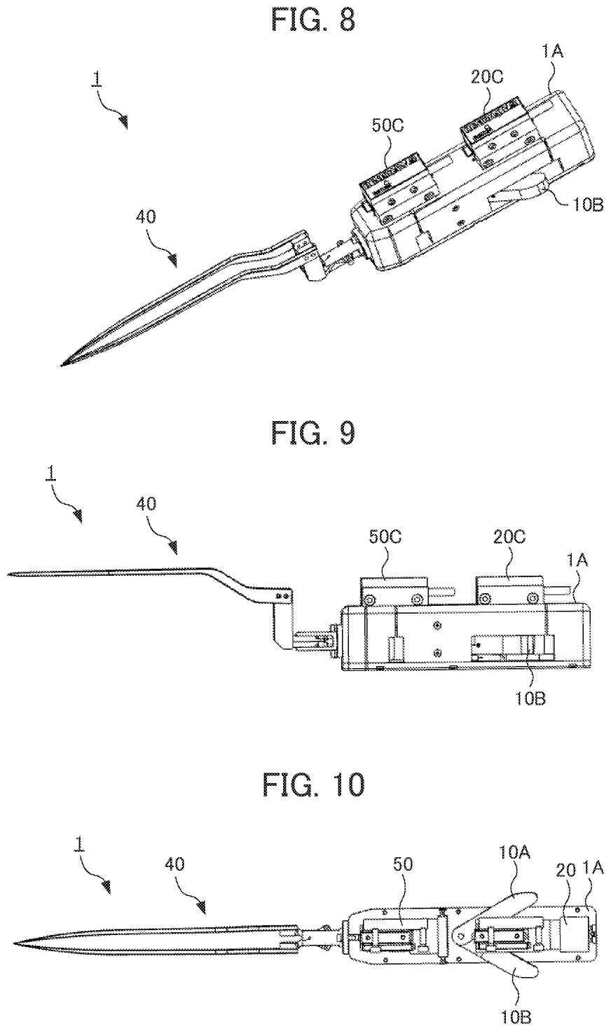

FIG. 8 is a schematic view of a modification of the first example of the device structure.

Note that FIG. 8 shows an external structure (is a perspective view) of the medical gripping device 1 according to the modification of the first example of the device structure.

FIG. 9 is a schematic side view of the medical gripping device 1 in the modification of the first example of the device structure. FIG. 10 is a schematic top view of the medical gripping device 1 in the modification of the first example of the device structure.

Note that FIG. 10 shows a main internal structure that can be seen through the upper surface of the housing 1A.

The modification shown in FIGS. 8 to 10 differs from the first example of the device structure shown in FIGS. 5 to 7 in that the gripping mechanism 40 having a distal-end shape that is the same as that of bayonet-type forceps is used. That is, in the modification shown in FIGS. 8 to 10, the gripping members 40A and 40B are disposed at positions that allow them to protrude beyond the upper surface of the housing 1A (offset positions with respect to a direction of extension of the housing 1A).

Due to such a structure, it is possible to provide a structure that allows an operator to easily see a grip object.

### [Second Example of Device Structure]

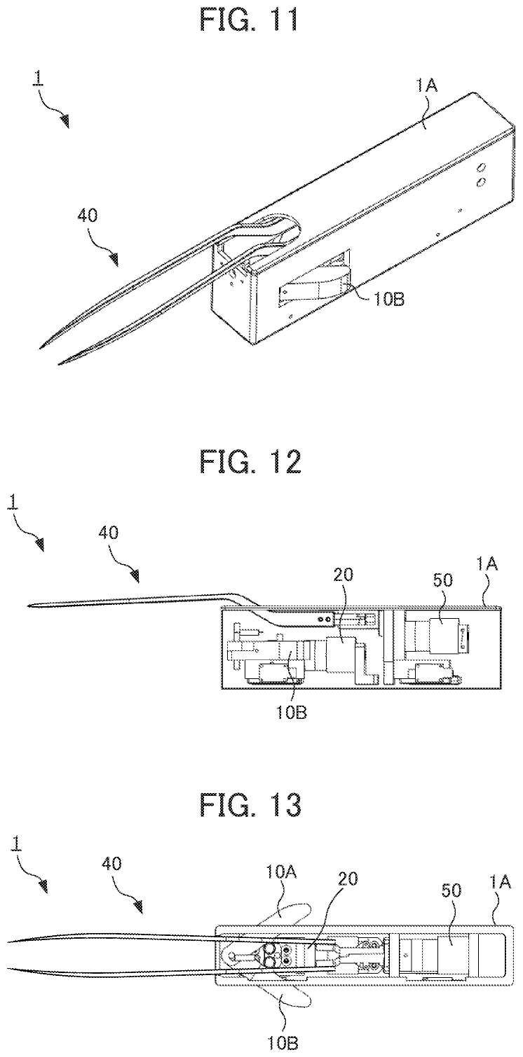

FIG. 11 is a schematic view of a second example of a device structure of the medical gripping device 1 according to the present invention.

Note that FIG. 11 shows an external structure (is a perspective view) of the medical gripping device 1 according to the second example of the device structure.

FIG. 12 is a schematic side view of the medical gripping device 1 in the second example of the device structure. FIG. 13 is a schematic top view of the medical gripping device 1 in the second example of the device structure.

Note that FIGS. 12 and 13 show a main internal structure that can be seen through a side surface and the upper surface of the housing 1A, respectively.

As shown in FIGS. 11 to 13, in the second example of the device structure, the gripping mechanism 40 is installed at one end of the housing 1A, and the gripping mechanism 40 extends in the longitudinal direction of the housing 1A from an open portion formed on one end side of the upper surface of the housing 1A. In the housing 1A, the operation unit 10 is installed on the one end side. Further, in the housing 1A, with respect to the operation unit 10, the reaction force actuator 20 is installed on the other end side that is opposite to the one end at which the gripping mechanism 40 is provided, and the gripping actuator 50 is installed further toward the other end side. In the second example of the device structure shown in FIGS. 11 to 13, in the housing 1A, the gripping mechanism 40 extends from the open portion in the upper surface via an upper portion of the operation unit 10 and an upper portion of the reaction force actuator (upper-surface side in the medical gripping device 1), and is connected to the gripping actuator 50. Note that the second example of the device structure shown in FIGS. 11 to 13 is an example in which the gripping mechanism 40 having a distal-end shape that is the same as that of bayonet-type forceps is provided.

In the case of such a structure, since the operation unit 10 can be installed at a position that is near the gripping mechanism 40, it becomes easier to set the center of gravity of the medical gripping device 1 at a location on which the back of a hand of an operator is placed.

In the second example of the device structure shown in FIGS. 11 to 13, the gripping members 40A and 40B are disposed at positions that allow them to protrude beyond the upper surface of the housing 1A (offset positions with respect to the direction of extension of the housing 1A).

Due to such a structure, it is possible to provide a structure that allows an operator to easily see a grip object.

### [Modification of Second Example of Device Structure]

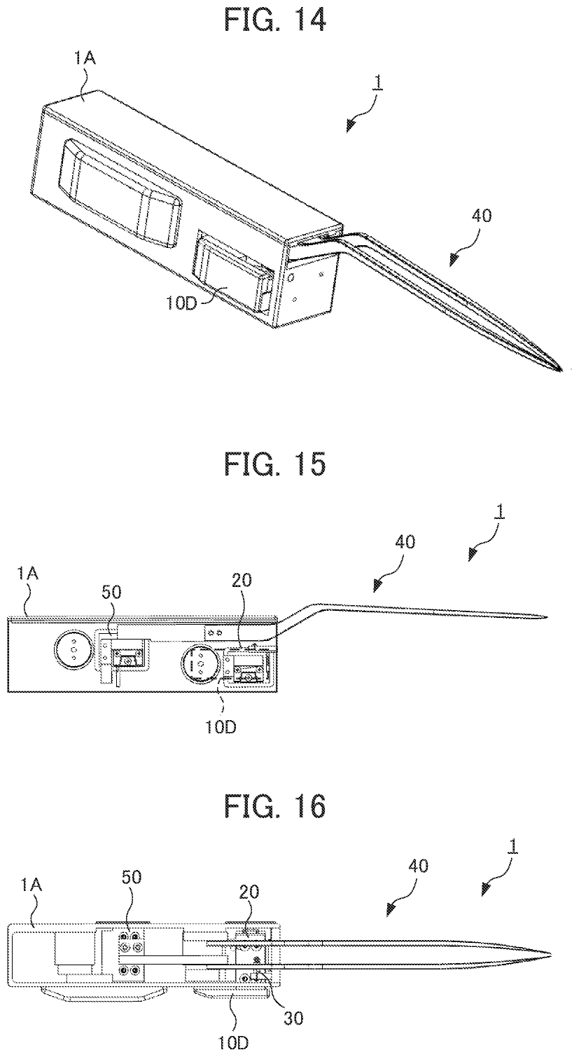

FIG. 14 is a schematic view of a modification of the second example of the device structure.

Note that FIG. 14 shows an external structure (is a perspective view) of the medical gripping device 1 according to the modification of the second example of the device structure.

FIG. 15 is a schematic side view of the medical gripping device 1 in the modification of the second example of the device structure. FIG. 16 is a schematic top view of the medical gripping device 1 in the modification of the second example of the device structure.

Note that FIGS. 15 and 16 show a main internal structure that can be seen through a side surface and the upper surface of the housing 1A [symbol] respectively.

The modification shown in FIGS. 14 to 16 differs from the second example of the device structure shown in FIGS. 11 to 13 in that the gripping mechanism 40 extends in the longitudinal direction of the housing 1A from an open portion formed in one end of the housing 1A and in that the mover 20A of the reaction force actuator 20 and the mover 50A of the gripping actuator 50 are installed in an orientation in which they move in the width direction of the housing 1A.

In the modification shown in FIGS. 14 to 16, the operation unit 10 includes, instead of the levers 10A and 10B, a push-in member 10D for a push-in operation at one of the side surfaces of the housing 1A.

In the modification shown in FIGS. 14 to 16, the reaction force actuator 20 is such that the stator 20B is fixed to a side surface on a side opposite to the push-in member 10D in the housing 1A, and a distal end of the mover 20A is connected to an inner surface (a surface on a side of the housing 1A) of the push-in member 10D. That is, by the push-in operation of the push-in member 10D, the mover 20A moves in a direction in which the mover 20A enters the stator 20B. One end of the restoring spring 30 is connected to the mover 20A of the reaction force actuator 20, and the other end of the restoring spring 30 is connected to the side surface in the housing 1A on a side at which the push-in member 10D is installed. The restoring spring 30 has a natural length when the push-in member is maximally protruded, and is brought into a stretched state the more the push-in member 10D is pushed in. Therefore, when the operation of the push-in member 10D is stopped, an elastic force of the restoring spring 30 causes the push-in member 10D to be restored to a maximally protruded state.

By causing the mover 50A of the gripping actuator 50 to move (linearly) in the width direction of the mover 50A, one of the gripping members 40A and 40B is moved away from or is moved toward the other of the gripping members 40A and 40B. In this case, a structure including, for example, slider-crank mechanisms for connecting the mover 50A and the gripping members 40A and 40B to each other is not required.

Due to such a structure, it is possible to reduce the number of mechanisms that are installed on the master side and the slave side, and to reduce the weight and the size of the medical gripping device 1.

In the modification shown in FIGS. 14 to 16, the gripping members 40A and 40B are disposed at positions that allow them to protrude beyond the upper surface of the housing 1A (offset positions with respect to the direction of extension of the housing 1A).

Due to such a structure, it is possible to provide a structure that allows an operator to easily see a grip object.

### [Operation]

Next, an operation of the medical gripping device 1 is described.

As described above, when an operator is to operate the medical gripping device 1, the operator operates the medical gripping device 1 with the index finger and the thumb gripping the levers 10A and 10B that are held between the index finger and the thumb and the medical gripping device 1 being placed on the back of the hand (first interdigital space).

In the embodiment, in an initial state (at the time of non-operation), the levers 10A and 10B of the operation unit 10 are in the maximally open state due to the elastic force of the restoring spring 30.

Therefore, when a power source of the medical gripping device 1 is turned on, the gripping members 40A and 40B are also in the maximally open state due to manual operation of the gripping mechanism 40 or an elastic force of a spring that corresponds to the restoring spring 30 and that is installed.

When the power source of the medical gripping device 1 is turned on, the position sensor 20C of the reaction force actuator 20 detects a position xₘ of the mover 20A, and the position sensor 50C of the gripping actuator 50 detects the position xₛ of the mover 50A. Then, the results of the detections are output to the control unit 60.

The control unit 60 multiplies the acceleration calculated from the position xₘ of the mover 20A by the master-side mass to calculate a force Fm that is output on the master side. Similarly, the control unit 60 multiplies the acceleration calculated from the position xₛ of the mover 50A by the slave-side mass to calculate a force Fₛ that is output by the slave robot.

Then, based on the position xₘ of the mover 20A, the force Fₘ that is output on the master side, the position xₛ of the mover 50A, and the force Fₛ that is output by the slave robot, the control unit 60 performs bilateral control in accordance with Formulas (7) and (8).

Therefore, the control is performed so that the position of the actuator of the master robot and the position of the actuator of the slave robot follow each other and the force that is output from the master robot and the reaction force that the slave robot is subjected to from an environment satisfy the action-reaction law.

Here, it is assumed that an operator positions a grip object between the gripping members 40A and 40B and the levers 10A and 10B perform a gripping operation on the master side.

Therefore, the bilateral control is performed as described above, and, in accordance with the operation amounts of the levers 10A and 10B, the gripping actuator 50 moves the mover 50A so as to close the gripping members 40A and 40B.

Then, when the gripping members 40A and 40B come into contact with the grip object, the reaction force Fₑₙᵥ is input to the gripping actuator 50 from an environment.

The reaction force Fₑₙᵥ is estimated by the reaction force observer and becomes data indicating the hardness of the grip object.

In the medical gripping device 1, since the control is performed so that the position of the actuator of the master robot and the position of the actuator of the slave robot follow each other and the force that is output from the master robot and the reaction force that the slave robot is subjected to from an environment satisfy the action-reaction law, the reaction force Fenv that has been input to the gripping actuator 50 is fed back as a force that is output by the reaction force actuator 20. As a result of the gripping members 40A and 40B coming into contact with the grip object, the positions that are determined in accordance with the gripping force are fed back as the positions that are output by the reaction force actuator 20 (the positions of the levers 10A and 10B).

At this time, if necessary, position or force scaling in the bilateral control is performed, and the magnitude of the positions or the magnitudes of the forces are increased or reduced and are transmitted.

Note that, when the positions and the forces are controlled so as to be fed back between the master robot and the slave robot, the disturbance observer compensates for the disturbance to stably control the positions and the forces.

Due to such an operation, in the medical gripping device 1, as a result of performing the bilateral control between the reaction force actuator 20 and the gripping actuator 50, the transmission of haptic sensation is performed between the operation for the gripping operation with respect to the operation unit 10 and the gripping operation of the gripping mechanism 40.

Due to the function of the reaction force observer, it is possible to acquire data about the hardness of the grip object from the parameters that are acquired in the bilateral control.

Further, by performing the bilateral control involving the position or force scaling, the magnitude of the positions or the magnitude of the forces can be increased or reduced and transmitted to the operator.

That is, according to the medical gripping device 1, it is possible to realize a medical gripping device that can be used as forceps and has more functions than forceps.

### [Effects]

Next, the effects of the medical gripping device 1 are described.

Note that, in the experiments below, the parameters shown in Table 1 are used.

TABLE 1

| | unit | |
|---|---|---|
| Position gain (Kₚ) | 1/s² | 10000 |
| Velocity gain (Kᵥ) | 1/s | 200 |
| Force gain (K_{f}) | | 1.0 |
| Cutoff frequency in DOB | rad/sec | 100 |
| Cutoff frequency in RFOB | rad/sec | 100 |
| Rotor inertia (Field mass) | kg | 0.035 |
| Thrust constant | N/A | 1.73 |

### [Experiment 1]

As Experiment 1, an experiment (tension spring rigidity measurement) that verifies whether the medical gripping device 1 according to the present invention is capable of measuring environmental rigidity was performed.

When the gripping mechanism 40 applies a force to an environment and is subjected to the reaction force Fₑₙᵥ, the relationship between the reaction force Fₑₙᵥ and an environmental rigidity kenv is expressed by Formula (11).

−Fₑₙᵥ=kₑₙᵥ(yₑₙᵥ−yₑₙᵥ0) (11)

Here, in order to accurately estimate the environmental rigidity, as a replacement of Formula (11), a method of dynamically estimating the environmental rigidity by using the amounts of change in position and force in a sample at time t and at time t-Δt before the time t is known. Here, kenv is expressed by Formula (12).

$$\begin{matrix}[ Math . 4 ] & \\ {κ}_{e n v} = - \frac{{F}_{e n v} ( t ) - {F}_{e n v} ( t - Δ t )}{{y}_{e n v} ( t ) - {y}_{e n v} ( t - Δ t )} & ( 12 )\end{matrix}$$

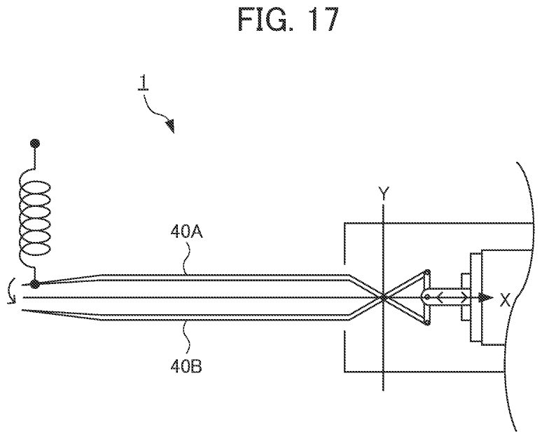

FIG. 17 is a schematic view of experimental conditions of Experiment 1.

In Experiment 1, the spring constant was used as an environment of a known tension spring.

Note that a spring constant k1 of the tension spring that is used here is 0.14×10³ [N/m].

As shown in FIG. 17, the tension spring was installed parallel to a Y axis, one end of the tension spring was fixed and the other end of the tension spring was held and pulled at a distal end of forceps. At this time, without performing scaling (α=1 [symbol] β=1), the reaction force that the gripping mechanism 40 was subjected to from the environment and the displacement of the gripping mechanism 40 were measured to calculate the environmental rigidity.

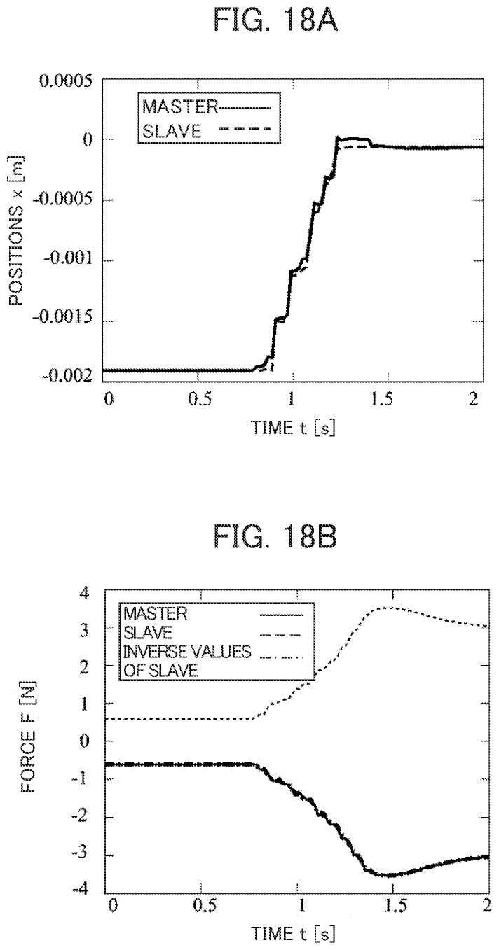

FIG. 18A shows time response measurement results of the positions of the actuator on the master side and the actuator on the slave side (here, voice coil motors were used). FIG. 18B shows inverse values of the reaction force that the slave gripping actuator 50 is subjected to from the environment.

The operation of gripping the tension spring is performed from t=0.9 [s] to t=1.3 [s]. Note that, for facilitating comparison, FIG. 18B shows the inverse values of the reaction force that the slave gripping actuator 50 is subjected to from the environment.

FIGS. 18A and 18B show that the medical gripping device 1 according to the present invention performs normal bilateral control.

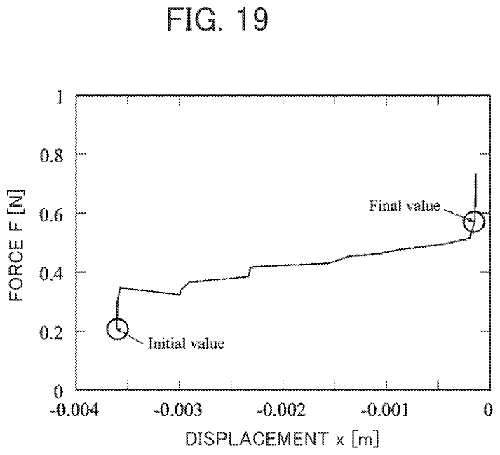

FIG. 19 shows changes in reaction force with respect to the displacement of the gripping mechanism 40 in Experiment 1.

In order to convert the response to the position or the force of the gripping actuator 50 to the response to the position and the force of the gripping mechanism 40, Formulas (1) and (3) to (6) were used.

From the calculation results, experimental values of the spring constant of the tension spring used as the environment are calculated.

As shown in FIG. 19, an initial value (yₑₙᵥ0 [symbol] Fₑₙᵥ0) was (−0.00361 [mm], 0.209 [N]).

The position of the spring used as the environment finally becomes constant.

Therefore, a final value (yₑₙᵥ [symbol] Fₑₙᵥ) becomes (−0.00013 [mm], 0.675 [N]).

From these values and Formula (12), an experimental value kₑₙᵥ′ of the environmental rigidity becomes 0.134×10³ [N/m].

This value is substantially the same as the applied spring constant k1, and the absolute error rate became 4.29 [%].

Therefore, it is possible to accurately estimate the environmental rigidity from the response to the force and the position in a motor space.

### [Experiment 2]

As Experiment 2, an experiment that distinguishes between models of normal brain cells and cancerous brain cells based on hardness was performed by using the medical gripping device 1 in the first example of the device structure.

Specifically, on the assumption that cancerous cells are extracted in brain surgery and based on discussions with surgeons, “soft tofu” was used as a model for normal brain cells and “firm tofu” was used as a model for cancerous brain cells.

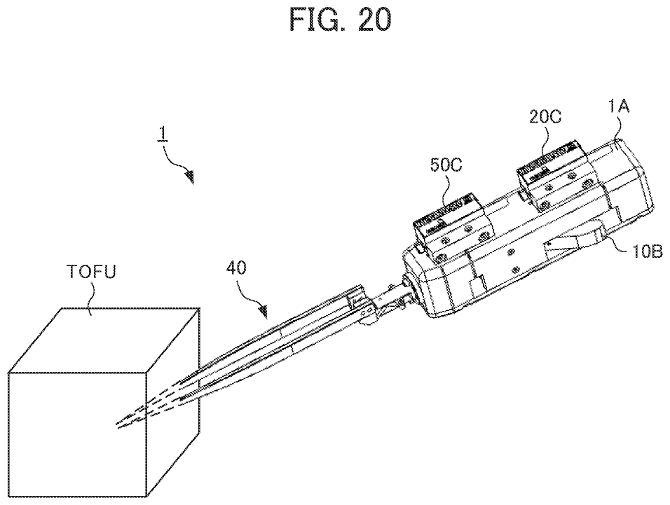

FIG. 20 is a schematic view of experimental conditions of Experiment 2.

As shown in FIG. 20, due to an operator operating the levers 10A and 10B on the master side, a tofu placed on the slave side is gripped via the gripping mechanism 40.

Two types of soft tofu and two types of firm tofu (numbered 1, 2, 3, and 4, with samples having odd numbers corresponding to the soft tofu and samples having even numbers corresponding to the firm tofu), that is, a total of four different types of tofu were used as grip objects.

With, as scaling magnifications, two types of combinations (α=1 [symbol] β=1) and (α=1 [symbol] β=1) being set, an experiment when a force had unity magnification (no scaling) and an experiment when a reaction force that was input to the slave robot was doubled and transmitted to the master robot (scaling in which the force was doubled) were performed.

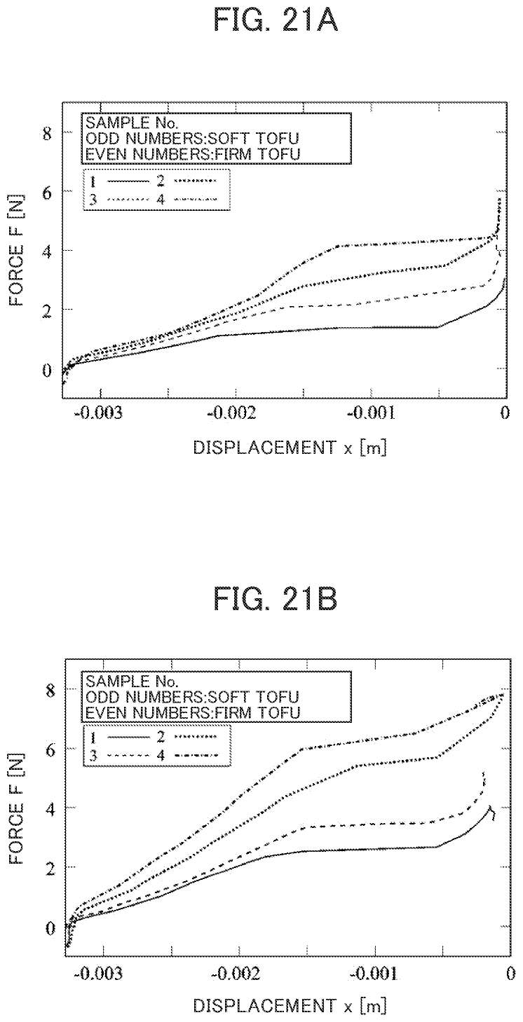

FIG. 21A shows the results of the experiment in which the samples are gripped without performing scaling. FIG. 21B shows the results of the experiment in which the samples are gripped by performing the scaling in which the force was doubled.

Note that FIGS. 21A and 21B show changes in force with respect to position in a motor space (the reaction force actuator 20) of the master robot.

As shown in FIGS. 21A and 21B, the environmental rigidity in a certain section can be visually read as an inclination of its graph. The larger the inclination, the larger a rigidity k, and a hard environment is provided.

FIG. 21A shows that, in the bilateral control in which the force has unity magnification, the hardnesses of the four types of tofu clearly differ from each other. In addition, the results show that the two types of firm tofu are harder than the two types of soft tofu, as a result of which the adequacy of Experiment 2 can be confirmed.

Further, FIG. 21B shows that, when scaling is performed to cause the master force to be twice the slave force, the hardnesses (the inclinations) clearly differ from each other.

Based on these results, it is possible to confirm the usability of the scaling method of the bilateral control.

Note that the present invention can be, for example, modified and improved as appropriate within a scope of the effects of the present invention, and is not limited to the embodiments above.

For example, in the first example of the device structure shown in FIGS. 5 to 7 and in the modification of the first example of the device structure shown in FIGS. 8 to 10, similarly to, for example, the second example of the device structure shown in FIGS. 11 to 13, a small encoder can be built in the housing 1A. In this case, it is possible to simplify the external shape of the housing 1A and to increase the operability of the medical gripping device 1.

In the embodiments above, a plurality of types of gripping mechanisms 40 of the medical gripping device 1 may be prepared and may be replaceable as attachments. In this case, for example, the gripping members 40A and 40B and the link members 41A and 41B can be formed into a unit as an attachment, and these components can be configured to be replaced by different types of such components. The control unit 60 accepts to set parameters in accordance with the structure of the replaced attachment, and, after the replacement, performs control that is in accordance with the structure of the newly mounted gripping mechanisms 40.

Therefore, it is possible to expand the use of the medical gripping device 1 and to improve convenience thereof.

In the embodiments above, a structure in which the gripping mechanism 40 is rotatable around a mounting shaft can be used. That is, the gripping mechanism 40 may be rotated around an axis in the longitudinal direction of the housing 1A and a gripping operation of an operator and a gripping operation of the gripping mechanism 40 may be performed so as to be staggered. In this case, the gripping actuator 50 and the gripping mechanism 40 are connected in a state of connection that allows an opening-closing operation even if they rotate around the axis.

Therefore, it is possible to perform a more suitable operation in accordance with the state of a grip object.

As described above, the medical gripping device 1 of the embodiments include the operation unit 10, the reaction force actuator 20, the gripping mechanism 40, the gripping actuator 50, the housing 1A, and the control unit 60.

The operation unit 10 is operated by a gripping operation of an operator.

The reaction force actuator 20 applies an operation reaction force to the operation unit 10.

The gripping mechanism 40 grips a grip object.

The gripping actuator 50 causes the gripping mechanism 40 to perform a gripping operation.

The housing 1A has the gripping mechanism 40 at one end and the operation unit 10 between the one end and the other end. The reaction force actuator 20 and the gripping actuator 50 are installed therein.

The control unit 60 controls a force and a position that are output by the gripping actuator 50 in the operation of the gripping mechanism 40 in accordance with the operation with respect to the operation unit 10, and controls a force and a position that are output by the reaction force actuator 20 in the operation of applying the operation reaction force to the operation unit 10 in accordance with a reaction from the grip object with respect to the gripping mechanism 40.

Therefore, the operator can operate, with a feeling of use that is similar to that of forceps of medical instruments, the medical gripping device of a form that is similar to forceps to which haptic sensation is transmitted by the bilateral control.

Consequently, according to the present invention, it is possible to realize a medical gripping device that can be used as forceps and has more functions than forceps.

In the medical gripping device 1, the position of the center of gravity of the entire device is set closer than the operation unit 10 to a side of the other end (the side opposite to the gripping mechanism 40).

Therefore, the operator is less likely to feel the weight of the medical gripping device 1 and the operability of the medical gripping device 1 can be increased.

The gripping mechanism 40 is disposed at an offset position with respect to the direction of extension of the housing 1A.

Therefore, it is possible to provide a structure that allows an operator to easily see a grip object.

In the medical gripping device 1, the gripping actuator 50 is installed closer than the reaction force actuator 20 to the other end side.

Therefore, since the operation unit 10 can be installed at a position that is near the gripping mechanism 40, it becomes easier to set the center of gravity of the medical gripping device 1 at a location on which the back of a hand of an operator is placed.

The medical gripping device 1 includes, at a location that is closer than the operation unit 10 to the other end side, the recess that receives a hand of an operator.

Therefore, it is possible to provide a structure having a shape that easily fits the shape of a hand of an operator.

The medical gripping device 1 includes an elastic member (the restoring spring 30) that causes an elastic force to be produced, the elastic force causing the operation unit 10 to be restored to its initial position at the time of non-operation.

Therefore, when an operator is not operating the operation unit 10, the operation unit 10 can be restored to its initial position and thus operability that is the same as that of forceps of medical instruments can be realized. In addition, a part of the operation reaction force can be provided by the elastic force of the elastic member.

The control unit 60 controls, by subtracting the elastic force of the elastic member (the restoring spring 30), a force that is output by the reaction force actuator 20 in the operation of applying the operation reaction force to the operation unit 10.

Therefore, even if the elastic member is provided, it is possible to add a suitable operation reaction force.

The control unit 60 adds the force of the reaction force actuator 20 to the elastic force of the elastic member (the restoring spring 30) to allow, at the operation unit 10, an elastic member having a spring constant differing from the spring constant of the elastic member to be felt.

Therefore, it is possible to adjust the medical gripping device 1 to a state that is easily operable by an operator, and to realize even higher operability thereof.

Note that, although, in the embodiments above, the position sensors 20C and 50C detect the positions of the movers 20A and 50A, respectively, it is not limited thereto. That is, the positions of the movers 20A and 50A can be detected by sensors and can also be estimated based on, for example, command values of the actuators.

The structure shown as the block diagram in the embodiments above can be realized as software in which the same functions are defined. In this case, a processor including the control unit 60 executes a program in which the functions in the block diagram in the embodiments above are described. In addition, the structure shown as the block diagram in the embodiments above can be realized as a hardware circuit, or can be realized as a combination of software and hardware.

That is, the process in the embodiments above can be realized by either hardware or software.

In other words, the functions that are capable of executing the process above only need to be provided in the medical gripping device 1, and thus what functional structure and what hardware structure are to be used for realizing the functions are not limited to the examples above.

When the process above is to be executed by software, a program that constitutes the software is installed into a computer from a network or a storage medium.

The storage medium that stores the program is constituted by, for example, a removable medium that is distributed separately from the device body, or a storage medium that is previously built in the device body. The removable medium is constituted by, for example, a magnetic disk, an optical disc, or a magneto-optical disk. The optical disc is constituted by, for example, a CD-ROM (Compact Disk-Read Only Memory), a DVD (Digital Versatile Disk), or a Blu-ray Disc (trademark). The magneto-optical disk is constituted by, for example, a MD (Mini-Disk). The storage medium that is previously built in the device body is constituted by, for example, a hard disk or ROM in which the program is stored.

### REFERENCE SIGNS LIST

1 medical gripping device, 1A housing, 10 operation unit, 10A, 10B lever, 10D push-in member, 11, 42 rotation shaft, 20 reaction force actuator, 20A, 50A mover, 20B, 50B stator, 20C, 50C position sensor, 30 restoring spring, 40 gripping mechanism, 40A, 40B gripping member, 41A, 41B link member, 50 gripping actuator, 60 control unit, 61 position-and-force control unit, 62 physical quantity acquisition unit.

1. A medical gripping device comprising:
an operation unit that is operated by a gripping operation of an operator;
a first actuator that applies an operation reaction force to the operation unit;
a gripping unit that grips a grip object;
a second actuator that causes the gripping unit to perform a gripping operation;
a housing that has the gripping unit at one end and the operation unit between the one end and the other end, the first actuator and the second actuator being installed in the housing; and
a control unit that controls a force and a position that are output by the second actuator in an operation of the gripping unit in accordance with an operation with respect to the operation unit, and controls a force and a position that are output by the first actuator in an operation of applying the operation reaction force to the operation unit in accordance with a reaction from the grip object with respect to the gripping unit.

2. The medical gripping device according to claim 1, wherein a position of a center of gravity of the device in an entirety thereof is set closer than the operation unit to a side of the other end.

3. The medical gripping device according to claim 1, wherein the gripping unit is disposed at an offset position with respect to a direction of extension of the housing.

4. The medical gripping device according to claim 1, wherein the second actuator is installed closer than the first actuator to a side of the other end.

5. The medical gripping device according to claim 1, further comprising, at a location that is closer than the operation unit to a side of the other end, a recess configured to receive a hand of the operator.

6. The medical gripping device according to claim 1, further comprising an elastic member that causes an elastic force to be produced, the elastic force causing the operation unit to be restored to an initial position at a time of non-operation.

7. The medical gripping device according to claim 6, wherein the control unit controls, by subtracting the elastic force of the elastic member, the force that is output by the first actuator in the operation of applying the operation reaction force to the operation unit.

8. The medical gripping device according to claim 6, wherein the control unit adds the force of the first actuator to the elastic force of the elastic member to allow, at the operation unit, an elastic member having a spring constant differing from a spring constant of the elastic member to be felt.

9. The medical gripping device according to claim 1, wherein the gripping unit is replaceable by a different type of member, and wherein, when the gripping unit has been replaced, the control unit performs control based on a parameter that is in accordance with a structure of the gripping unit that has been mounted.

10. The medical gripping device according to claim 1, wherein the gripping unit is rotatable around a mounting shaft.
